(19) United States
(12) Patent Application Publication
(10) Pub. No.: US 2009/0069363 A1
(43) Pub. Date: Mar. 12, 2009

(54) Therapeutic Agent for Constipation

(75) Inventors: Tsutomu Suzuki, Kanagawa (JP); Takuko Sawada, Osaka (JP); Yasunobu Ishihara, Osaka (JP)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: SHIONOGI & CO. LTD. A LEGAL ENTITY OF JAPAN, OSAKA (JP)

(21) Appl. No.: 11/792,893

(22) PCT Filed: Dec. 13, 2005
(86) PCT No.: PCT/JP2005/022822

§ 371 Date: Oct. 3, 2007

(30) Foreign Application Priority Data
Dec. 14, 2004 (JP) 2004-360966
Feb. 4, 2005 (JP) 2005-028927
Apr. 8, 2005 (JP) 2005-111912
Oct. 11, 2005 (JP) 2005-296045

(51) Int. Cl. A61K 31/485; C07D 489/00; A61P 1/10

(52) U.S. Cl. 514/282; 546/44

(57) ABSTRACT

A therapeutic and/or prophylactic agent for constipation induced by a compound having an opioid μ receptor agonist activity, which agent comprises as an effective ingredient a compound having an opioid δ receptor antagonist activity, e.g., a compound of Formula (I):

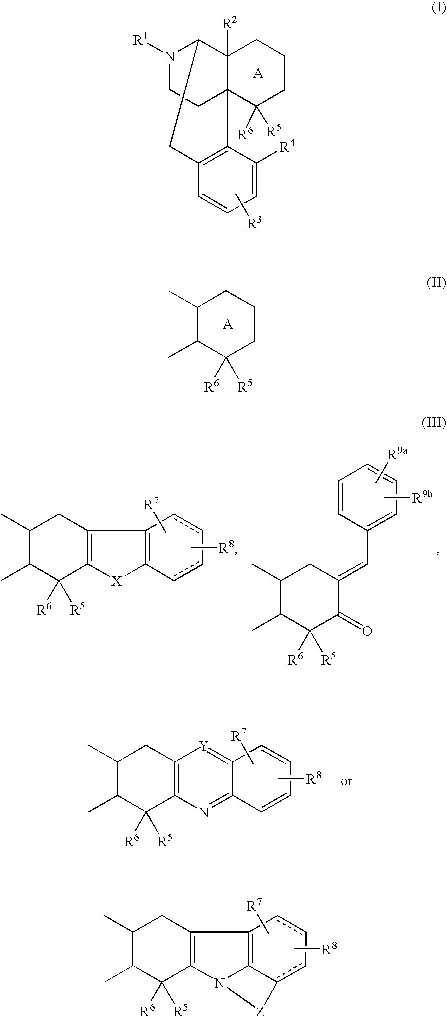

(wherein R¹ represents hydrogen, lower alkyl, cycloalkyl lower alkyl or the like; R² and R³ independently represent hydrogen, hydroxy or the like; R⁴ is hydrogen, hydroxy or the like; R⁵ is hydrogen; R⁴ and R⁵ may optionally form —O— or the like; R⁶ represents hydrogen, lower alkyl or the like (wherein X represents —O— or —N(R¹⁰)— or the like; R⁷, R⁸, R^{9a} and R^{9b} independently represent hydrogen, lower alkyl, lower alkoxycarbonyl or the like; r represents an integer of 0 to 5; Y represents —CH— or the like; Z represents a crosslinkage composed of 2 to 5 atoms) or a pharmaceutically acceptable salt thereof or a solvate of either.

### TECHNICAL FIELD

The present invention relates to a therapeutic and/or prophylactic agent for constipation in which opioid μ receptor is involved, especially, which is induced by a compound having a μ agonist activity.

### BACKGROUND ART

Opioid μ receptor agonists such as morphine are used as very effective analgesics for patients suffering from cancer pain. However, they induce strong vomiting, nausea, constipation, urinary retention, itching and the like as side effects. Although various antiemetics and anti-constipation drugs are clinically used, none of them exhibits sufficient effects, so that an excellent agent for reducing the side effects is demanded for the improvement of QOL of the patients.

As the pharmaceuticals whose indication is dysfunction of digestive tract or constipation caused by administration of a nacrotic analgesic, methylnaltrexone bromide (MNTX), alvimopan and the like, which are opioid μ receptor antagonists, are now being developed.

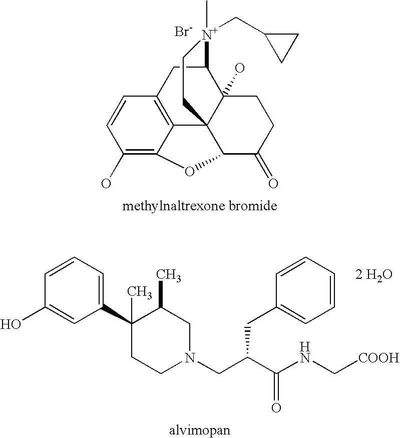

Patent Literature 1 discloses that naloxone, naltrexone and the like are effective for amelioration of dysfunction of gastrointestinal active function.

Patent Literatures 2 to 4 and Non-patent Literature 1 disclose that MNTX and derivatives thereof, naloxone, N-methylnaloxone and the like are effective for amelioration of side effects induced by an opioid, and constipation is listed as an example of the side effects.

Patent Literatures 5 to 7 disclose that piperidine-N-alkylcarboxylate derivatives which are opioid μ antagonists are effective for irritable bowel syndrome, constipation, ileus and the like.

Although the compound (I) of the present invention and analogues thereof are disclosed in Patent Literatures 8 to 20 and Non-patent Literatures 2 and 3, none of them discloses the therapeutic or prophylactic effect thereof for constipation.
Patent Literature 1: International Patent Publication WO83/03197
Patent Literature 2: International Patent Publication WO99/22737
Patent Literature 3: International Patent Publication WO01/032180
Patent Literature 4: International Patent Publication WO98/25613
Patent Literature 5: Japanese Laid-open Patent Application (Kokai) No. H5-97806
Patent Literature 6: International Patent Publication WO01/037785
Patent Literature 7: International Patent Publication WO01/42207
Patent Literature 8: International Patent Publication WO89/00995
Patent Literature 9: International Patent Publication WO95/31463
Patent Literature 10: International Patent Publication WO94/07896
Patent Literature 11: International Patent Publication WO97/11948
Patent Literature 12: International Patent Publication WO02/42309
Patent Literature 13: International Patent Publication WO2004/007503
Patent Literature 14: International Patent Publication WO98/31684
Patent Literature 15: International Patent Publication WO94/14445
Patent Literature 16: International Patent Publication WO91/07966
Patent Literature 17: U.S. Pat. No. 6,271,239
Patent Literature 18: International Patent Publication WO95/13071
Patent Literature 19: International Patent Publication WO93/21188
Patent Literature 20: U.S. Pat. No. 6,476,044.
Non-patent Literature 1: Journal of Pharmacology and Experimental Therapeutics, 300 (1), 118-123, (2002)
Non-patent Literature 2: Heterocycles 45, 2109-2112 (1997)
Non-patent Literature 3: Journal of Medicinal Chemistry 41, 4177-4180 (1998)
   Problems which the Invention Tries to Solve

An object of the present invention is to provide a therapeutic and/or prophylactic agent for constipation induced by a compound having an opioid μ receptor agonist activity.

### Means for Solving the Problems

The present invention provides:
(1) A therapeutic and/or prophylactic agent for constipation in which opioid μ receptor is involved, the agent comprising a compound having an opioid δ receptor antagonist activity;
(2) A therapeutic and/or prophylactic agent for constipation induced by a compound having an opioid μ receptor agonist activity, the agent comprising a compound having an opioid δ receptor antagonist activity;
(3) The therapeutic and/or prophylactic agent for constipation according to (1) or (2) above, wherein the compound having the opioid δ receptor antagonist activity has a higher affinity to opioid δ receptor than to opioid μ receptor;
(4) The therapeutic and/or prophylactic agent for constipation according to (1) or (2) above, wherein the compound having the opioid δ receptor antagonist activity is represented by Formula (I):

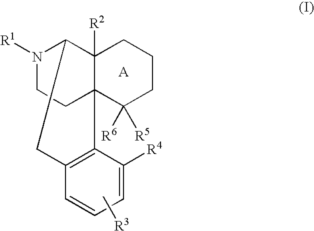

(wherein R¹ represents hydrogen, lower alkyl, cycloalkyl lower alkyl, cycloalkenyl lower alkyl, lower alkenyl, aryl, aryl lower alkyl, furyl lower alkyl or thienyl lower alkyl;

R² and R³ independently represent hydrogen, hydroxy, lower alkoxy, lower alkenyloxy, aryl lower alkoxy, aryl lower alkenyloxy, acyloxy or lower alkoky lower alkoxy;

R⁴ represents hydrogen, hydroxy, lower alkoxy or acyloxy;

R⁵ represents hydrogen;

R⁴ and R⁵ may optionally together form —O—, —S— or —CH₂—;

R⁶ represents hydrogen, lower alkyl, lower alkenyl, hydroxy lower alkyl, lower alkoxy lower alkyl, lower alkoxycarbonyl lower alkyl, aryl lower alkyl, aryl lower alkenyl, carboxy or lower alkoxycarbonyl;

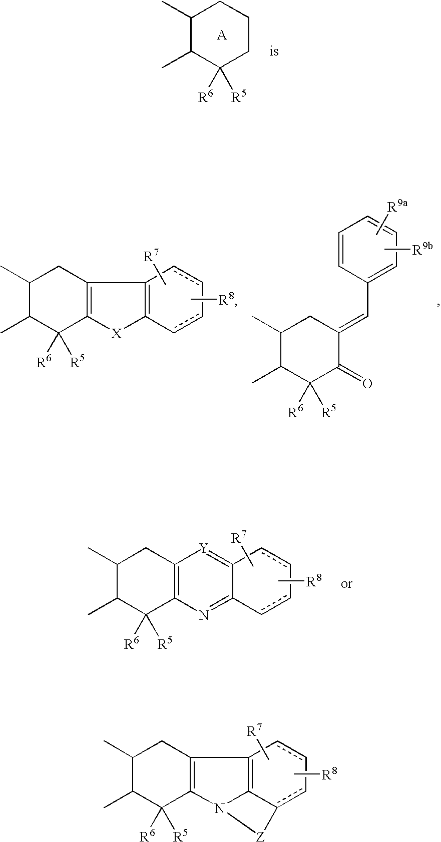

(wherein X represents —O—, —S—, —CH═CH— or —N(R¹⁰)—;

R⁷, R⁸, R^{9a} and R^{9b} independently represent hydrogen, halogen, nitro, lower alkyl, hydroxy, lower alkoxy, halogeno lower alkyl, hydroxy lower alkyl, halogeno lower alkoxy, hydroxy lower alkoxy, cyano, phenyl, isothiocyanato, SR¹¹, SOR¹¹, SO₂R¹¹, (CH₂)ᵣOR¹¹, (CH₂)ᵣCOOR¹¹, SO₂NR¹²R¹³, CONR¹²R¹³, (CH₂)ᵣNR¹²R¹³ or (CH₂)ᵣN(R¹²)COR¹³;

R⁷ and R⁸ may optionally bind to adjacent carbon atoms in the ring to form a ring together with the carbon atoms, which ring may have a substituent(s);

broken lines represent presence or absence of a bond, and in cases where the broken lines represent absence of the bond, R⁷ and R⁸ may optionally together form ═O;

r represents an integer of 0 to 5;

R¹⁰ represents hydrogen, lower alkyl, lower alkenyl, aryl lower alkyl, aryl lower alkenyl, acyl, lower alkylsulfonyl, arylsulfonyl, aryl lower alkylsulfonyl or acyl;

Y represents —N— or —CH—;

Z represents a crosslinkage composed of 2 to 5 atoms;

R¹¹ represents hydrogen or lower alkyl;

R¹² and R¹³ independently represent hydrogen, lower alkyl or cycloalkyl lower alkyl) or a pharmaceutically acceptable salt thereof or a solvate of either (hereinafter referred to as “compound (I)”);
(5) The therapeutic and/or prophylactic agent according to )(4) above, wherein R¹ is cycloalkyl lower alkyl;

R² and R³ are hydroxy;

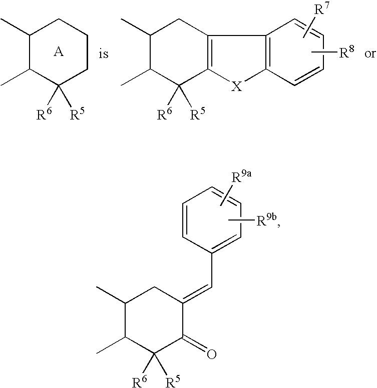

R⁴ and R⁵ together form —O—;

R⁶ is hydrogen;

R⁷, R⁸, R^{9a} and R^{9b} independently are hydrogen, lower alkyl, carboxy or lower alkoxycarbonyl;

R¹⁰ is hydrogen or lower alkyl; and
(6) The therapeutic and/or prophylactic agent according to any one of (2) to (5) above, wherein the compound having the opioid μ receptor agonist activity is morphine, oxycodone or a pharmaceutically acceptable salt thereof.

The present invention also provides:
(7) Use of a compound having an opioid δ receptor antagonist activity for the therapy and/or prophylaxis of constipation in which opioid μ receptor is involved;
(8) Use of a compound having an opioid 6 receptor antagonist activity for the therapy and/or prophylaxis of constipation induced by a compound having an opioid μ receptor agonist activity;
(9) Use of the compound represented by Formula (I) recited in (4) above or a pharmaceutically acceptable salt thereof or a solvate of either for the therapy and/or prophylaxis of constipation in which opioid μ receptor is involved;
(10) Use of a compound represented by Formula (I) recited in (4) above or a pharmaceutically acceptable salt thereof or a solvate of either for the therapy and/or prophylaxis of constipation induced by a compound having an opioid μ receptor agonist activity;
(11) A therapeutic and/or prophylactic method for constipation in which opioid μ receptor is involved, the method comprising administering a compound having an opioid δ receptor antagonist activity;
(12) A therapeutic and/or prophylactic method for constipation induced by a compound having an opioid μ receptor agonist activity, the method comprising administering a compound having an opioid δ receptor antagonist activity;
(13) A therapeutic and/or prophylactic method for constipation in which opioid μ receptor is involved, the method comprising administering the compound represented by Formula (I) recited in (4) above or a pharmaceutically acceptable salt thereof or a solvate of either;

(14) A therapeutic and/or prophylactic method for constipation induced by a compound having an opioid μ receptor agonist activity, the method comprising administering the compound represented by Formula (I) recited in (4) above or a pharmaceutically acceptable salt thereof or a solvate of either; and

(15) An analgesic comprising a compound having an opioid μ receptor agonist activity in combination with the compound represented by Formula (I) recited in (4) above or a pharmaceutically acceptable salt thereof or a solvate of either in an amount effective for the therapy and/or prophylaxis of constipation induced by the compound having the opioid μ receptor agonist activity.

### Effects of the Invention

The compounds having an opioid δ receptor antagonist activity (hereinafter referred to as “the compound of the present invention”) have a therapeutic and/or prophylactic activity against constipation in which opioid μ receptor is involved, especially against the constipation induced by a compound having an agonist activity, and are useful as an agent for reducing side effects in the patients who are to receive or who are receiving a compound having an opioid μ receptor agonist activity.

### BEST MODE FOR CARRYING OUT THE INVENTION

In the present description, the term “halogen” includes fluorine, chlorine, bromine and iodine.

The above-described explanation is equally applied to the halogen moiety in “halogeno lower alkyl” and “halogeno lower alkoxy”.

The term “lower alkyl” includes linear and branched alkyl groups having 1 to 10, preferably 1 to 6, more preferably 1 to 3 carbon atoms. Examples thereof include methyl, ethyl, n-propyl, isopropyl, n-butyl, isobutyl, sec-butyl, tert-butyl, n-pentyl, isopentyl, neopentyl, hexyl, isohexyl, n-heptyl, isoheptyl, n-octyl, isooctyl, n-nonyl, n-decyl and the like.

The above-described explanation about the “lower alkyl” is equally applied to the lower alkyl moiety in “halogeno lower alkyl”, “hydroxy lower alkyl”, “cycloalkyl lower alkyl”, “cycloalkenyl lower alkyl”, “lower alkoxycarbonyl lower alkyl”, “aryl lower alkyl”, “furyl lower alkyl”, “thienyl lower alkyl”, “aryl lower alkylsulfonyl”, “lower alkoxy lower alkyl”, “lower alkylsulfonyl”, “lower alkoxy”, “lower alkoxy lower alkoxy”, “halogeno lower alkoxy”, “hydroxy lower alkoxy”, “aryl lower alkoxy” and “lower alkoxy carbonyl”.

The term “lower alkenyl” includes linear and branched alkenyl groups having one or more double bonds at an optional site(s) and having 2 to 10, preferably 2 to 8, more preferably 3 to 6 carbon atoms. Examples thereof include vinyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl and the like.

The above-described explanation about the “lower alkenyl” is equally applied to the lower alkenyl moiety in “aryl lower alkenyl”, “lower alkenyloxy” and “aryl lower alkenyloxy”.

The term “aryl” includes phenyl, naphthyl, anthryl and phenanthryl, and phenyl is especially preferred.

The above-described explanation about the “aryl” is equally applied to the aryl moiety in “aryl lower alkyl”, “aryl lower alkylsulfonyl”, “aryl lower alkoxy”, “aryl lower alkenyl”, “aryl lower alkenyloxy” and “arylsulfonyl”.

The term “acyl” includes linear and branched chain aliphatic acyl groups having 1 to 10, preferably 1 to 6, more preferably 1 to 4 carbon atoms, cyclic aliphatic acyl groups having 4 to 9, preferably 4 to 7 carbon atoms and aroyl. Examples thereof include formyl, acetyl, propionyl, butyryl, isobutyryl, valeryl, pivaloyl, hexanoyl, acryloyl, propioloyl, methacryloyl, crotonoyl, cyclopropylcarbonyl, cyclohexylcarbonyl, cyclooctylcarbonyl, benzoyl and the like.

The chain aliphatic acyl may be substituted with an aryl group(s), lower alkyl aryl group(s) and/or the like. The cyclic aliphatic acyl and aroyl may be substituted with a lower alkyl group(s).

The above-described explanation about the “acyl” is equally applied to the acyl moiety in “acyloxy”.

The term “cycloalkyl” means a carbocyclic group having 3 to 8, preferably 3 to 6 carbon atoms. Examples thereof include cyclopropyl, cyclobutyl, cyclopentyl, cyclohexyl, cycloheptyl, cyclooctyl and the like.

The above-described explanation about the “cycloalkyl” is equally applied to the cycloalkyl moiety in “cycloalkyl lower alkyl”.

The term “cycloalkenyl” includes those having one or more double bonds at an optional site(s) in the ring of the above-described cycloalkyl group. Examples thereof include cyclopropenyl, cyclobutenyl, cyclopentenyl, cyclohexenyl, cycloheptynyl, cyclooctynyl, cyclohexadienyl and the like.

The above-described explanation about the “cycloalkenyl” is equally applied to the cycloalkenyl moiety in “cycloalkenyl lower alkyl”.

The phrase “R⁷ and R⁸ may optionally bind to adjacent carbon atoms in the ring and to form a ring together with the carbon atoms, which ring may optionally have a substituent(s)” means that

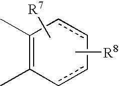

forms, for example,

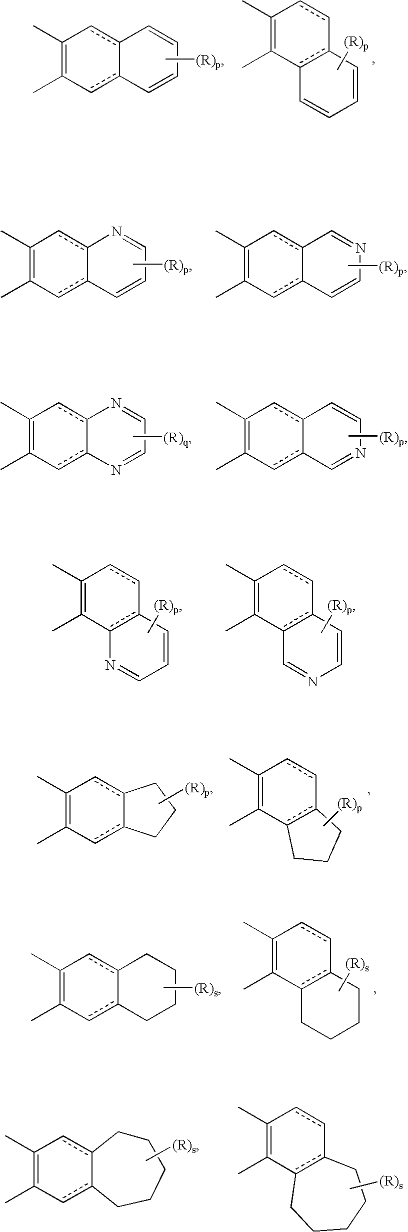

or the like
(wherein R represents lower alkyl, lower alkoxy, acyl, hydroxy lower alkyl, SR¹¹, SOR¹¹, SO₂R¹¹, (CH₂)ᵣOR¹¹, (CH₂)ᵣCOOR¹¹, SO₂NR¹²R¹³, CONR¹²R¹³, (CH₂)ᵣNR¹²R¹³ or (CH₂)ᵣ(R¹²)COR¹³; p represents an integer of 0 to 3; q represents an integer of 0 to 2; s represents an integer of 0 to 4; and other symbols have the same meanings as described above. In cases where p, q and r are not less than 2, Rs may be the same or different).

The phrase “Z represents a crosslinkage composed of 2 to 5 atoms” means that Z is, for example, —(CR^{9a}R^{9b})₂—, —(CR^{9a}R^{9b})₃—, —(CR^{9a}R^{9b})₄—, —(CR^{9a}R^{9b})₅—, —(CR^{9a}R^{9b})₂O—, —(CR^{9a}R^{9b})₂S—, (CR^{9a}R^{9b})₂N(R¹⁰)—, —O(CR^{9a}R^{9b})₂—, —S(CR^{9a}R^{9b})₂—, —N(R¹⁰)(CR^{9a}R^{9b})₂— (wherein R^{9a}, R^{9b} and R¹⁰ have the same meanings as described above, and in cases where a plurality of R^{9a}s and R^{9b}s exist, R^{9a}s may be different, and R^{9b}s may be different) or the like.

In the present description, the term “solvate” includes, for example, solvates with organic solvents, hydrates and the like. In cases where a hydrate is formed, the compound may be coordinated with an optional number of water molecules.

Compound (I) includes pharmaceutically acceptable salts. Examples thereof include salts with an alkaline metal (such as lithium, sodium or potassium), an alkaline earth metal (such as magnesium or carcium), an ammonium; an organic base, or an amino acid; and salts with an inorganic acid (such as hydrochloric acid, sulfuric acid, nitric acid, hydrobromic acid, phosphoric acid or hydroiodic acid) or organic acid (such as acetic acid, citric acid, lactic acid, tartaric acid, oxalic acid, maleic acid, fumaric acid, mandelic acid, glutaric acid, malic acid, benzoic acid, phthalic acid, benzenesulfonic acid, p-toluenesulfonic acid, methanesulfonic acid or ethanesulfonic acid). Salts with hydrochloric acid, phosphoric acid, tartaric acid, methanesulfonic acid or the like are especially preferred. These salts can be formed by an ordinary method.

Compound (I) is not restricted to a specific isomer, but includes all possible isomers and racemates.

Preferred examples of R¹ include hydrogen, lower alkyl, cycloalkyl lower alkyl, lower alkenyl, aryl lower alkyl, furyl lower alkyl and thienyl lower alkyl, and cyclopropylmethyl is especially preferred.

Preferred examples of R² include hydrogen, hydroxy, lower alkoxy, aryl lower alkoxy and acyloxy, and hydroxy is especially preferred.

Preferred examples of R³ include hydrogen, hydroxy, lower alkoxy, aryl lower alkoxy and acyloxy, and hydroxy is especially preferred.

As for R⁴ and R⁵, those wherein R⁴ is hydrogen, hydroxy, lower alkoxy or acyloxy, and R⁵ is hydrogen, and those wherein R⁴ and R⁵ together form —O— or —S— are preferred.

Preferred examples of R⁶ include hydrogen, lower alkyl, carboxy and lower alkoxycarbonyl, and hydrogen is especially preferred.

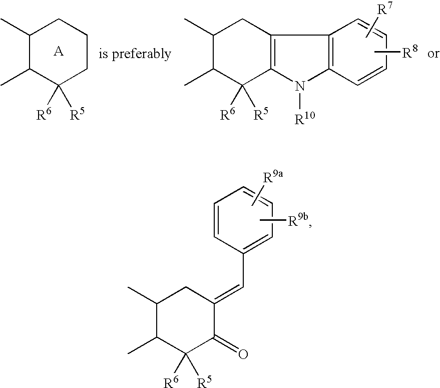

especially wherein R⁷ and R⁸ independently are hydrogen, halogen, nitro, C₁-C₃ alkyl, hydroxy, C₁-C₃ alkoxy, halogeno C₁-C₃ alkoxy, hydroxy C₁-C₃ alkyl, cyano, phenyl, isothiocyanato, SR¹⁴, SOR¹⁴, SO₂R¹⁴, (CH₂)ᵣOR¹⁴, (CH₂)ᵣCOOR¹⁴, SO₂NR¹⁵R¹⁶, CONR¹⁵R¹⁶ or (CH₂)ᵣNR¹⁵R¹⁶ (wherein R¹⁴ is C₁-C₃ alkyl, R¹⁵ and R¹⁶ independently are hydrogen or C₁-C₃ alkyl, and r is an integer of 0 to 5), or R⁷ and R⁸ together with the adjacent carbon atoms to which they are bound to form benzene ring, cyclopentane ring or cyclohexane ring; R^{9a} and R^{9b} independently are hydrogen or C₁-C₃ alkyl; and R¹⁰ is hydrogen or C₁-C₃ alkyl.

The compound (I) of the present invention can be produced by the methods such as those described in the above-described Patent Literatures 8, 9, 11 and 15, and in Non-patent Literature 2.

The “compound having an opioid δ receptor antagonist activity” may be any compound as long as it has a high affinity to δ receptor than to opioid receptors such as μ receptor and δ receptor (for example, the affinity to δ receptor is not less than 10 times, preferably not less than 20 times, more preferably not less than 30 times higher than the affinities to other opioid receptors), and has a δ receptor antagonist activity.

Examples thereof include 7-benzilidenenaltrexone (BNTX), [D-Ala², Leu⁵, Cys⁶] enkephalin (DALCE), naltriben, naltrindole 5′-isothiocyanate (5′- NTII), H-Tyr-Tic-Phe-Phe-OH(TIPP), naltrindole, N,N-diallyl-Tyr-Aib-Aib-Phe-Leu-OH (ICI174,864), (N,N-bisallyl)-Tyr-Gly-Gly-Ψ-(CH₂S)-Phe-Leu-OH (ICI-154,129) and the like. Preferably, the compound having an opioid δ receptor antagonist activity is the above-described compound (I), a salt thereof, or a solvate of compound (I).

The term “constipation in which opioid μ receptor is involved” means the constipation induced by taking a compound having an opioid μ receptor agonist activity. Examples of the “compound having an opioid μ receptor agonist activity” include morphine, oxycodon, fentanyl, methadone, codeine, dihydrocodeine, hydromorphone, levorphanol, meperidine, propoxyphene, dextropropoxyphene and tramadol, as well as pharmaceutically acceptable salts thereof. The therapeutic and/or prophylactic agent according to the present invention is especially effective when the compound is morphine or oxycodon or a pharmaceutically acceptable salt thereof.

The compound of the present invention exhibits high amelioration effect against small intestine transit inhibition, the inhibitory action being induced by a μ receptor agonist administered to a patient suffering from a disease accompanying pain (e.g., cancer pain (pain due to bone metastasis, compression of nerve, intracranial hypertension, infiltration into soft tissue, pain induced by constipation or twitching; pain of an internal organ, muscle or fascia; pain of lumbar or the vicinity of shoulder joint; chronic postsurgical pain), AIDS or the like), without substantially reducing the analgesic effect of the μ receptor agonist. Therefore, the compound of the present invention is useful as a therapeutic and/or propylactic agent against not only constipation, but also against irritable bowel syndrome or the like.

Especially, compound (I) has features such as high oral availability, low brain penetration, low toxicity and high stability in human plasma, and is very useful as a pharmaceutical.

The compound of the present invention may be administered before, after or simultaneously with the administration of the compound having the opioid μ receptor agonist activity.

The interval between the administration of the two drugs is not restricted. For example, in cases where the compound is administered after administration of the compound having the opioid μ receptor agonist activity, the compound of the present invention well functions if it is administered immediately after to about 3 days after, preferably immediately after to about 1 day after the administration of the compound having the opioid μ receptor agonist activity. In cases where the compound is administered before administration of the compound having the opioid μ receptor agonist activity, the compound of the present invention well functions if it is administered just before to about 1 day before, preferably just before to about 12 hours before the administration of the compound having the opioid μ receptor agonist activity.

When administering the compound of the present invention as a therapeutic or prophylactic agent for constipation, another therapeutic or prophylactic agent(s) for constipation may be used in combination. For example, a stimulant laxative(s) (such as sennoside and sodium picosulfate), and then an osmotic laxative(s) (lactulose) or a saline laxative(s) (magnesium oxide) may be administered in combination.

When the compound of the present invention is administered to human as a therapeutic or prophylactic agent, the compound may be formulated in the form of powder, granules, tablets, capsules, balls, liquid or the like and may be administered orally, or the compound may be formulated in the form of injection solution, suppository, transdermal formulation, inhalant or the like and may be administered parenterally. The compound of the present invention may be formulated into a pharmaceutical preparation by mixing an effective amount of the compound of the present invention with a preferable additive(s) for pharmaceuticals such as vehicle(s), binder(s), wetting agent(s), disintegrator(s) and/or lubricant(s) according to its formulation.

The compound of the present invention may be in the form of a combination with the compound having the opioid μ receptor agonist activity and/or other therapeutic or prophylactic agent(s) for constipation, as well as various additives for pharmaceuticals as required.

Although the dose varies depending on the state of the disease, administration route and the age or body weight of the patient, the dose for an adult for oral administration is usually 1 μ g to 10 g/day, preferably 0.1 to 2000 mg/day, and the dose for parenteral administration is usually 0.1 μ g to 1 g/day, preferably 0.01 to 200 mg/day.

The present invention will now be described in more detail referring to examples and test examples. However, the present invention is not restricted thereto.

### EXAMPLES

### Test Example 1

### Influence on Morphine-Induced Inhibitory Action against Transport Competence of Gastrointestinal Tract

### 1) Test Compounds

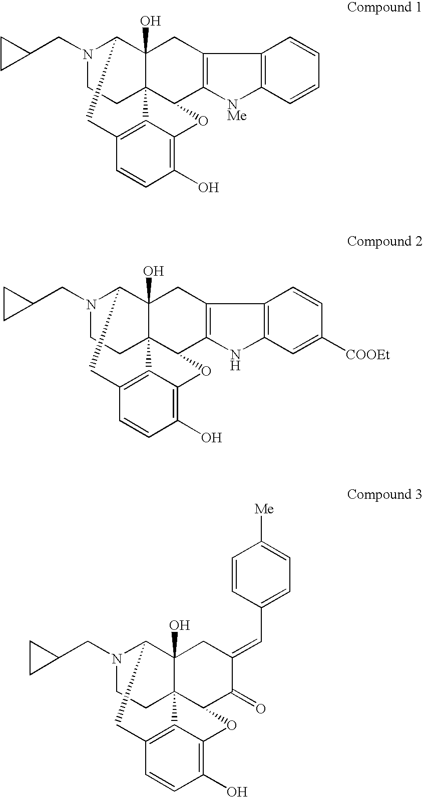

### 2) Preparation Method of Test Solutions

### Test Solutions for Oral Administration:

After weighing each test compound, 0.5% methylcellulose (methylcellulose: Wako Pure Chemicals, water for injection: Otsuka Pharmaceutical Factory) was added, and the obtained mixture was well stirred to prepare solutions containing the test compound at final concentrations of 3 mg/mL, 1 mg/mL and 0.3 mg/mL, respectively.

### Test Solutions for Subcutaneous Administration:

After weighing each test compound, 5% xylitol (Kylit injection (registered trademark), Otsuka Pharmaceutical Factory) was added and the mixture was well stirred to prepare solutions containing the test compound at a final concentration of 1 mg/mL. The solutions having concentrations of 0.3 mg/mL and 0.1 mg/mL, respectively, were prepared by diluting the solution having the concentration of 1 mg/mL.

### 3) Other Drugs

Morphine hydrochloride (Sankyo, hereinafter referred to as “morphine”)

Preparation Method: After weighing, physiological saline was added and the mixture was well stirred to prepare a solution having a concentration of 0.3 mg/mL.

### 4) Animals

Crj:CD-1(ICR) mouse (SPF, male, 5-week old, body weight: 25.6-32.0 g (oral administration), 26.3-33.0 g (subcutaneous administration), Charles River Laboratories Japan)

### 5) Rearing Conditions

Temperature: 20-26° C.
Humidity: 35-75%
Lighting Hours: 12 hours/day (7 to 19 o'clock)
Number of Ventilation: 15-25 times/hour
Feed: Solid Feed F-2 (Funabashi Farms), free feeding
Drinking: tap water, free feeding

### 6) Test Groups

### Oral Administration:

Control Group: 8 animals; Solvent-administered Group: 8 animals; Compound 1: 3, 10 and 30 mg/kg Groups: each 8 animals; Compound 2: 3, 10 and 30. mg/kg Groups: each 8 animals; Compound 3: 3, 10 and 30 mg/kg Groups: each 8 animals

### Subcutaneous Administration

Control Group: 8 animals, Solvent-administered Group: 8 animals; Compound 1: 3, 10 and 30 mg/kg Groups: each 8 animals; Compound 2: 3, 10 and 30 mg/kg Groups: each 8 animals; Compound 3: 3, 10 and 30 mg/kg Groups: each 8 animals

### 7) Administration

Administration volume: 10 mL/kg
Administration Method: Oral administration was carried out using a disposable syringe and an oral feeding tube.

Subcutaneous administration was carried out using a disposable syringe and a disposable injection needle.
Administration Frequency: single

### 8) Test Procedure

Mice fasted overnight from the evening on one day before the test were used. Each test compound was administered, and 15 minutes later, morphine (3 mg/kg) was subcutaneously administered. 30 minutes after the administration of morphine, carbon powder (a suspension containing 5% of carbon powder suspended in 10% gum Arabic) was orally administered to each mouse in an amount of 0.1 mL, and the transfer rate (the distance which the carbon powder reached/the distance between the pylorus to cecum opening×100) was measured at 30 minutes after the administration of carbon powder. To the solvent-administered group, a solvent (methylcellulose or xylitol) was administered in place of the test substance solution. To the control group, none of the test substance, morphine and the solvent was administered and the transfer rate of the carbon powder was measured.

### 9) Results

The results obtained by oral administration of the test substances are shown in Table 1 and in FIG. 1.

TABLE 1

| | Dose of | | Transfer Rate |
|---|---|---|---|
| | Test | Dose of | of Carbon |
| | Substance | Morphine | Powder (%) |
| Control Group | — | — | 57 ± 1 |
| Solvent-administered | — | 3 mg/kg(s.c.) | 25 ± 2 |
| Group (Solvent: 0.5% | | | |
| methylcellulose) | | | |
| Compound 1-administered | 3 mg/kg | 3 mg/kg(s.c.) | 38 ± 2** |
| Group | 10 mg/kg | 3 mg/kg(s.c.) | 35 ± 2** |
| | 30 mg/kg | 3 mg/kg(s.c.) | 40 ± 2** |
| Compound 2-administered | 3 mg/kg | 3 mg/kg(s.c.) | 43 ± 2** |
| Group | 10 mg/kg | 3 mg/kg(s.c.) | 38 ± 2** |
| | 30 mg/kg | 3 mg/kg(s.c.) | 52 ± 3** |

**<0.01

The results obtained by subcutaneous administration of the test substances are shown in Table 2 and in FIG. 2.

TABLE 2

| | Dose of | | Transfer Rate |
|---|---|---|---|
| | Test | Dose of | of Carbon |
| | Substance | Morphine | Powder (%) |
| Control Group | — | — | 60 ± 5 |
| Solvent-administered | — | 3 mg/kg(s.c.) | 27 ± 1 |
| Group | | | |
| (Solvent: 5% xylitol) | | | |
| Compound 1-administered | 1 mg/kg | 3 mg/kg(s.c.) | 41 ± 2** |
| Group | 3 mg/kg | 3 mg/kg(s.c.) | 43 ± 3** |
| | 10 mg/kg | 3 mg/kg(s.c.) | 46 ± 3** |
| Compound 2-administered | 1 mg/kg | 3 mg/kg(s.c.) | 35 ± 3 |
| Group | 3 mg/kg | 3 mg/kg(s.c.) | 53 ± 3** |
| | 10 mg/kg | 3 mg/kg(s.c.) | 55 ± 2** |
| Compound 3-administered | 1 mg/kg | 3 mg/kg(s.c.) | 32 ± 2 |
| Group | 3 mg/kg | 3 mg/kg(s.c.) | 43 ± 1** |
| | 10 mg/kg | 3 mg/kg(s.c.) | 53 ± 3** |

**<0.01

As seen from the above-described results, compound (I) exhibited antagonistic action against the gastrointestinal transit inhibition, which was induced by administration of morphine.

### Example 1

### Synthesis of 17-cyclopropylmethyl-6,7-didehydro-4,5α-epoxy-3,14β-dihydroxy-6′-carboxy-6,7-2′,3′-indolomorphinan (4)

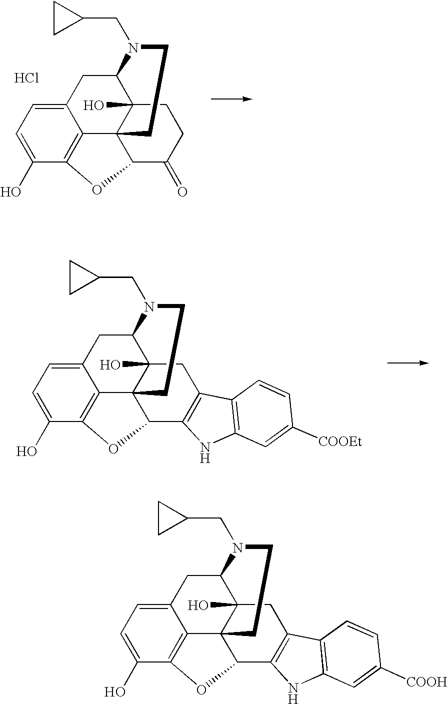

(First Step) 17-cyclopropylmethyl-6,7-didehydro-4,5α-epoxy-3,14β-dihydroxy-6′-ethoxycarbonyl-6,7-2′,3′-indolomorphinan

Known naltrexone hydrochloric acid salt (500 mg, 1.32 mmol) and o-hydrazinobenzoic acid (221 mg, 1.46 mmol) were suspended in 3 ml of ethanol, and the suspension was heated at 50° C. with stirring. To the mixture, methanesulfonic acid (0.86 mL, 13.2 mmol) solution in 2 mL of ethanol was slowly added dropwise for 10 minutes. After completion of the dropwise addition, the mixture was stirred for 2 hours under reflux. After allowing the mixture to cool to room temperature, saturated aqueous sodium hydrogen carbonate solution and ethyl acetate were added to the reaction solution. The organic layer was separated and washed with water and then with saturated brine. After drying the organic layer over sodium sulfate, the solvent was evaporated. The residue was purified by silica gel column chromatography (chloroform:methanol=99:1) to obtain 379 mg (59%) of the captioned compound as pale yellow solid.

NMR (300 MHz, CDCl₃) δ 0.14-0.18 (m, 2H), 0.55-0.59 (m, 2H), 0.89 (m, 1H), 1.41 (t, 3H, J=6.9 Hz), 1.75 (d, 1H, J=11.4 Hz), 2.20-2.91 (m, 8H), 3.10 (d, 1H, J=18.6 Hz), 3.38 (d, 1H, J=6.3 Hz), 4.38 (q, 2H, J=6.9 Hz), 5.5 (br s, 1H), 5.69 (s, 1H), 6.46 (d, 1H, J=8.1 Hz), 6.55 (d, 1H, J=8.1 Hz), 7.34 (d, J=8.4 Hz), 7.67 (d, J=8.4 Hz), 7.92 (s, 1H), 8.36 (s, 1H).
(Second Step) 17-cyclopropylmethyl-6,7-didehydro-4,5α-epoxy-3,14β-dihydroxy-6′-carboxy-6,7-2′,3′-indolomorphinan

To a solution of the compound (654 mg, 1.20 mmol) obtained in Step 1 in methanol (2.4 mL), 2 mol/L aqueous sodium hydroxide solution (2.4 mL) was added, and the mixture was stirred for 1 hour under reflux. After allowing the reaction solution to cool to room temperature, the reaction solution was diluted with methanol, and its pH was adjusted to 6.0 with dilute hydrochloric acid. The precipitated crystals were collected by filtration, washed with water and dried to obtain 534 mg (97%) of the captioned compound as colorless crystals.

NMR (300 MHz, d6-DMSO) δ 0.14-0.18 (m, 2H), 0.48-0.54 (m, 2H), 0.90 (m, 1H), 1.59 (d, 1H, J=11.7 Hz), 2.09-2.82 (m, 8H), 3.07 (d, 1H, J=18.6 Hz), 5.55 (s, 1H), 6.49 (d, 1H, J=7.8 Hz), 6.52 (d, 1H, J=7.8 Hz), 7.42 (d, J=8.4 Hz), 7.55 (dd, J=1.5, 8.4 Hz), 7.97 (d, J=1.5 Hz, 1H), 8.98 (br s, 1H), 11.54 (s, 1H).

### Test Example 2

### Influence on Morphine-Induced Small Intestine Transit Inhibition

### 1) Preparation of Test Meal (Pigment)

Using aqueous 0.5 w/v % Evans blue solution, 2.5 w/v % carboxymethylcellulose salt solution was prepared and used as the test meal.

### 2) Animals Used

Male Wistar rats (Crj. Wistar, Charles River Laboratories Japan, 6 to 7-week old) were used. The rats were fasted from not less than 20 hours before the beginning of the test, and water was given ad libitum.

### 3) Test Substance and Medium

The compound (I-1) synthesized in Example 1 was dissolved in a solvent (DMAA/Solutol/5% meglumine=15/15/70)
DMAA: N,N-dimethylacetamide (Kanto Chemical)
Solutol: Solutol (registered trademark) HS15 (BASF)
Meglumine: D(−)-N-methylglucamin (Merck)

Morphine hydrochloric acid salt (Dainippon Pharma) was dissolved in normal saline.

All of the test substance, solvent and morphine were administered in avolume of 2 mL/kg.

### 4). Test Method

Each test substance in an amount of 0.03, 0.1, 0.3, 1 or 3 mg/kg (Test Substance—administered Groups) or the above-described solvent (Solvent-administered Group) was subcutaneously administered, and 75 minutes later, 3 mg/kg of morphine was subcutaneously administered to all groups. To the control group, the above-described solvent was subcutaneously administered, and 75 minutes later, normal saline was administered.

The test meal in an amount of 2 mL/rat was orally administered at 30 minutes after the administration of morphine. 15 minutes after the administration of the test meal (at 120 minutes after the administration of the test substance), the portion from the vicinity of cardia in the esophagus to the ileocecum was extirpated. The distance between the cardia and the ileocecum (full length of small intestine), and the distance up to the tip of the pigment (moving distance of the pigment) were measured.

### 5) Data Processing

Transport Rate (%)=(Moving Distance of Pigment (cm)/Full Length of Small Intestine (cm))×100

M.P.E.(%)={(A(%)−B(%)}/(C(%)−B(%))}×100
(A: Transport Rate (%) in Small Intestine of Each Individual in Test Substance-administered Group;
B: Mean Transport Rate (%) in Small Intestine of Solvent-administered Group
C: Mean Transport Rate (%) in Small Intestine of Control Group

ED₅₀ was calculated using % MPE, by the inverse estimation of the regression of SAS program taking the value of the control group as 100%. As the significant test, Dunnett's test was used.

### 6) Results

The compound of the present invention exhibited antagonistic action against the morphine-induced small intestine transit inhibition, and the ED₅₀ value was 0.29 mg/kg.

### Formulation Example 1

Granules comprising the following components are prepared:

Components

| | | |
|---|---|---|
| Compound of Formula (I) | 10 | mg |
| Lactose | 700 | mg |
| Corn Starch | 274 | mg |
| HPC-L | 16 | mg |
| | 1000 | mg |

The compound of Formula (I) and lactose are made to pass through a 60-mesh sieve. Corn starch is made to pass through a 120-mesh sieve. These are mixed with a V-shaped mixer. An aqueous HPC-L (low viscosity hydroxypropylcellulose) solution is added to the mixed powder, and the resulting mixture is subjected to kneading, granulation (extrusion granulation, pore size 0.5 to 1 mm) and drying. The obtained dried granules are made to pass through a vibrating sieve (12/60-mesh) to obtain granules.

### Formulation Example 2

Granules for capsulation containing the following components are prepared:

Components

| | | |
|---|---|---|
| Compound of Formula (I) | 15 | mg |
| Lactose | 90 | mg |
| Corn Starch | 42 | mg |
| HPC-L | 3 | mg |
| | 150 | mg |

The compound of Formula (I) and lactose are made to pass through a 60-mesh sieve. Corn starch is made to pass through a 120-mesh sieve. These are mixed and an aqueous HPC-L solution is added to the mixed powder, and the resulting mixture is subjected to kneading, granulation and drying. After regulating the particle size of the dried granules, 150 mg thereof is filled in a hardness 4 gelatin capsule.

### Formulation Example 3

Tablets containing the following components are prepared:

Components

| | | |
|---|---|---|
| Compound of Formula (I) | 10 | mg |
| Lactose | 90 | mg |
| Microcrystalline Cellulose | 30 | mg |
| CMC-Na | 15 | mg |
| Magnesium Stearate | 5 | mg |
| | 150 | mg |

The compound of Formula (I), lactose, microcrystalline cellulose and CMC-Na (carboxymethylcellulose sodium salt) were made to pass through a 60-mesh sieve and mixed. The mixed powder is mixed With magnesium stearate to obtain mixed powder for tableting. The mixture is directly subjected to tablet making to obtain 150 mg of tablets.

### Formulation Example 4

The following components were mixed under heat, and the mixture was sterilized to obtain an injection solution.

| | | |
|---|---|---|
| Compound of the Present Invention | 3 | mg |
| Nonionic Surfactant | 15 | mg |
| Purified Water for Injection | 1 | ml |

### INDUSTRIAL AVAILABILITY

The compound of the present invention can be a pharmaceutical useful for the therapy or prophylaxis of constipation.

### BRIEF DESCRIPTION OF THE DRAWINGS

FIG. 1 is a graph showing the influence on the gastrointestinal transit when the compound of the present invention was orally administered.

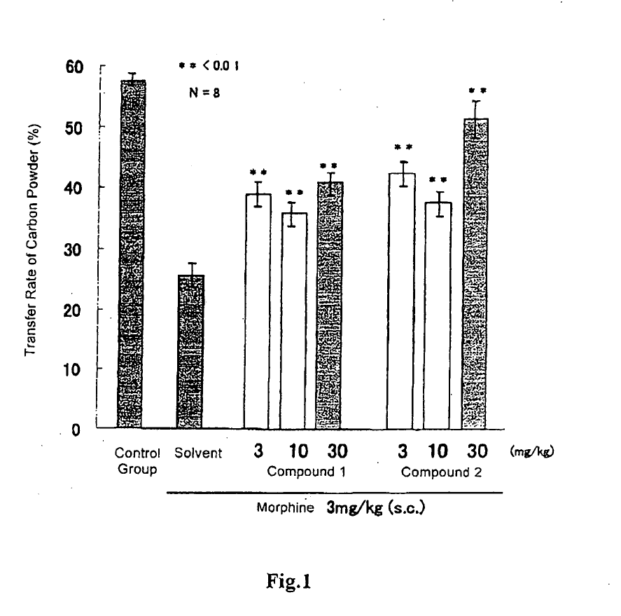

FIG. 2 is a graph showing the influence on the gastrointestinal transit when the compound of the present invention was subcutaneously administered.

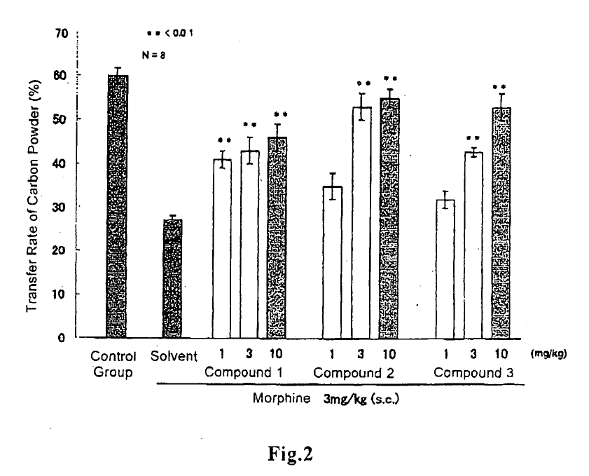

1. A therapeutic and/or prophylactic agent for constipation in which opioid μ receptor is involved, said agent comprising a compound having an opioid δ receptor antagonist activity.

2. A therapeutic and/or prophylactic agent for constipation induced by a compound having an opioid μ receptor agonist activity, said agent comprising a compound having an opioid δ receptor antagonist activity.

3. The therapeutic and/or prophylactic agent for constipation according to claim 1 or 2, wherein said compound having the opioid δ receptor antagonist activity has a higher affinity to opioid δ receptor than to opioid μ receptor.

4. The therapeutic and/or prophylactic agent for constipation according to claim 1 or 2, wherein said compound having the opioid δ receptor antagonist activity is represented by Formula (I): 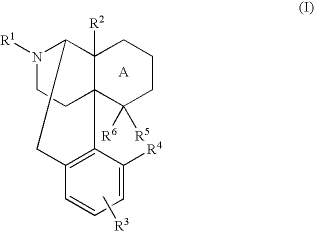
(wherein R¹ represents hydrogen, lower alkyl, cycloalkyl lower alkyl, cycloalkenyl lower alkyl, lower alkenyl, aryl, aryl lower alkyl, furyl lower alkyl or thienyl lower alkyl;
R² and R³ independently represent hydrogen, hydroxy, lower alkoxy, lower alkenyloxy, aryl lower alkoxy, aryl lower alkenyloxy, acyloxy or lower alkoxy lower alkoxy;
R⁴ represents hydrogen, hydroxy, lower alkoxy or acyloxy, R⁵ represents hydrogen;
R⁴ and R⁵ may optionally together form —O—, —S— or —CH₂—;
R⁶ represents hydrogen, lower alkyl, lower alkenyl, hydroxy lower alkyl, lower alkoxy lower alkyl, lower alkoxycarbonyl lower alkyl, aryl lower alkyl, aryl lower alkenyl, carboxy or lower alkoxycarbonyl; 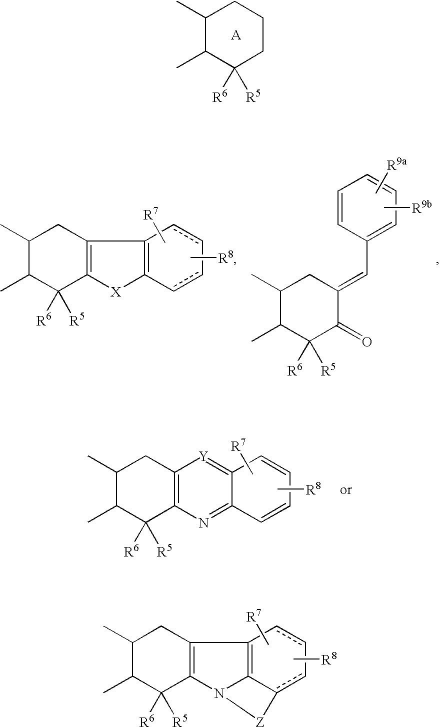
(wherein X represents —O—, —S—, —CH═CH— or —N(R¹⁰)—;
R⁷, R⁸, R^{9a} and R^{9b} independently represent hydrogen, halogen, nitro, lower alkyl, hydroxy, lower alkoxy, halogeno lower alkyl, hydroxy lower alkyl, halogeno lower alkoxy, hydroxy lower alkoxy, cyano, phenyl, isothiocyanato, SR¹¹, SOR¹¹, SO₂R¹¹, (CH₂)ᵣOR¹¹, (CH₂)ᵣCOOR¹¹, SO₂NR¹²R₁₃, CONR¹²R¹³, (CH₂)ᵣNR¹²R¹³ or (CH₂)ᵣN(R²)COR¹³;
R⁷ and R⁸ may optionally bind to adjacent carbon atoms in the ring to form a ring together with the carbon atoms, which ring may have a substituent(s);
broken lines represent presence or absence of a bond, in cases where the broken lines represent absence of the bond and, R⁷ and R⁸ may optionally together form ═O;
r represents an integer of 0 to 5;
R¹⁰ represents hydrogen, lower alkyl, lower alkenyl, aryl lower alkyl, aryl lower alkenyl, acyl, lower alkylsulfonyl, arylsulfonyl, aryl lower alkylsulfonyl or acyl;
Y represents —N— or —CH—;
Z represents a crosslinkage composed of 2 to 5 atoms;
R¹¹ represents hydrogen or lower alkyl;
R¹² and R¹³ independently represent hydrogen, lower alkyl or cycloalkyl lower alkyl) or a pharmaceutically acceptable salt thereof or a solvate of either.

5. The therapeutic and/or prophylactic agent according to claim 4, wherein R¹ is cycloalkyl lower alkyl;
R² and R³ are hydroxy; 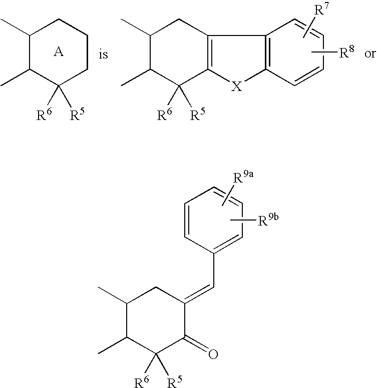
R⁴ and R⁵ together form —O—;
R⁶ is hydrogen;
R⁷, R⁸, R^{9a} and R^{9b} independently are hydrogen, lower alkyl, carboxy or lower alkoxycarbonyl;
R¹⁰ is hydrogen or lower alkyl.

6. The therapeutic and/or prophylactic agent according to any one of claims 2 to 5, wherein said compound having the opioid μ receptor agonist activity is morphine, oxycodone or a pharmaceutically acceptable salt thereof.

7. Use of a compound having an opioid δ receptor antagonist activity for the therapy and/or prophylaxis of constipation in which opioid μ receptor is involved.

8. Use of a compound having an opioid δ receptor antagonist activity for the therapy and/or prophylaxis of constipation induced by a compound having an opioid μ receptor agonist activity.

9. Use of the compound represented by Formula (I) recited in claim 4 or a pharmaceutically acceptable salt thereof or a solvate of either for the therapy and/or prophylaxis of constipation in which opioid μ receptor is involved.

10. Use of a compound represented by Formula (I) recited in claim 4 or a pharmaceutically acceptable salt thereof or a solvate of either for the therapy and/or prophylaxis of constipation induced by a compound having an opioid μ receptor agonist activity.

11. A therapeutic and/or prophylactic method for constipation in which opioid μ receptor is involved, said method comprising administering a compound having an opioid δ receptor antagonist activity.

12. A therapeutic and/or prophylactic method for constipation induced by a compound having an opioid μ receptor agonist activity, said method comprising administering a compound having an opioid δ receptor antagonist activity.

13. A therapeutic and/or prophylactic method for constipation in which opioid μ receptor is involved, said method comprising administering the compound represented by Formula (I) recited in claim 4 or a pharmaceutically acceptable salt thereof or a solvate of said compound or the pharmaceutically acceptable salt thereof.

14. A therapeutic and/or prophylactic method for constipation induced by a compound having an opioid μ receptor agonist activity, said method comprising administering the compound represented by Formula (I) recited in claim 4 or a pharmaceutically acceptable salt thereof or a solvate of either.

15. An analgesic comprising a compound having an opioid μ receptor agonist activity in combination with the compound represented by Formula (I) recited in claim 4 or a pharmaceutically acceptable salt thereof or a solvate of either in an amount effective for the therapy and/or prophylaxis of constipation induced by said compound having the opioid μ receptor agonist activity.
